(19) United States
(12) Patent Application Publication
(10) Pub. No.: US 2008/0009713 A1
(43) Pub. Date: Jan. 10, 2008

(54) MEDICAL PELVIC POSITIONING AND TRACKING DEVICE

(76) Inventor: Gregor Tuma, Munich (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/749,175

(22) Filed: May 16, 2007

Related U.S. Application Data
(60) Provisional application No. 60/803,296, filed on May 26, 2006.

(30) Foreign Application Priority Data
May 16, 2006 (EP) 06010122

(51) Int. Cl. A61B 19/00; A61B 5/05; A47B 7/00

(52) U.S. Cl. 600/424; 606/130; 5/621

(57) ABSTRACT

A medical pelvic positioning and tracking device includes at least one pelvic support element arranged on a patient support structure and a magnetic field generator operative to generate a magnetic field for an electromagnetic tracking system. The magnetic field generator is arranged in or on the at least one pelvic support element.

### RELATED APPLICATION DATA

This application claims priority of U.S. Provisional Application No. 60/803,296 filed on May 26, 2006, which is incorporated herein by reference in its entirety.

### FIELD OF THE INVENTION

The invention relates to medical navigation and, more particularly, to a medical pelvic positioning and tracking device.

### BACKGROUND OF THE INVENTION

When performing medical treatments in the pelvic and/or hip region of a patient, for example when placing implants, it is highly advantageous if the treatments can be performed with the assistance of a medical navigation system that provides image assisted surgery. Preferably, in image-assisted surgery of the pelvic and/or hip region, the pelvis of the patient is precisely positioned and immovably fixed. Further, a position of the pelvis is preferably ascertainable at least on the basis of defined planes. Ascertaining positional data for parts of the body or instruments is commonly referred to as “tracking”, and provides positional input values for medical navigation.

Optical tracking systems are known, for example, from DE 196 39 615 A1, the contents of which is hereby incorporated by reference in its entirety. In addition, electromagnetic tracking systems are also known, for example, from EP 1 502 620 A1 and U.S. Pat. No. 6,332,089 B1, each of which also are incorporated by reference in its entirety. Electromagnetic tracking systems are highly suitable for applications in which a continuous line of sight from a camera to a reference marker cannot always be maintained, in particular when parts within a patient's body are to be tracked. Electromagnetic tracking systems typically use a separately arranged external magnetic field generator in order to localize and track small coils that are attached to parts of the patient's body and/or to surgical instruments. For tracking purposes, magnetic field generators have a restricted field volume. Further, the generators have to be accommodated in a sterile environment. Systems also exist in which disposable field generators are directly attached to the patient or are held by hand in the vicinity of the tracking coils.

A patient positioning device comprising a localizing system for the pelvic plane based on optical tracking is known from US 2004/0254586 A1.

### SUMMARY OF THE INVENTION

A medical pelvic positioning and tracking device is provided that includes one or more pelvic support elements arranged on a patient support structure (e.g., a table or the like for placement of the patient), and a magnetic field generator for generating a magnetic field for an electromagnetic tracking system. The magnetic field generator is arranged in or on one of the pelvic support elements.

The device thus provides an arranging or mounting point for the magnetic field generator, wherein the point is optimally located since the one or more pelvic support elements are situated in the immediate vicinity of the pelvis. This enables a magnetic field to be generated in the treatment region that is sufficiently strong, homogenous and stable.

Attaching the magnetic field generator in the immediate vicinity of the pelvis prevents the magnetic field generator or its supply lines from obstructing the medical work space. In other words, the arrangement allows the magnetic field generator to be positioned stationary in the immediate vicinity of the instruments or parts of the patient's body to be tracked. The magnetic field generator thus is optimally integrated into the treatment and/or positioning environment, and only a single device is necessary in order to perform electromagnetic tracking and to simultaneously fix the pelvis, since the pelvic support element can be used in most pelvic operations.

The device is simple to use, can be left unsterile, and can be used repeatedly. Further, the proximity of the magnetic field generator to the operation and tracking region makes tracking itself very precise, even within a relatively small field or tracking volume.

In a preferred embodiment, the device comprises multiple, in particular three, pelvic support elements, wherein one pelvic support element comprises the magnetic field generator, and magnetic coil tracking devices are arranged on the other pelvic support elements. The magnetic coil tracking devices can in principle be passive resonance coil tracking devices or active magnetic coil tracking devices that include an integrated or external power supply and/or control unit.

The magnetic field generator can be arranged, sterilely enclosed, in or on a pelvic support element, in particular in the interior of a pelvic support element.

The pelvic support elements can include support element holders on which the pelvic support elements are adjustably arranged. The position of the pelvic support elements (e.g., the three support elements) relative to each other can be manually adjusted, and their position relative to an origin point (e.g., an origin of the magnetic field generator) can be known by tracking the coils. Knowing the adjustment and/or the precise position of the support elements relative to each other can be utilized for subsequently referencing bones or instruments relative to said position. In this case, it is possible to guide the supply lines for the magnetic field generator or active magnetic coil tracking devices on or in the holders. This is advantageous in that even magnetic coil tracking means that are supplied with power or controlled via cables can be laid in the operating area such that the cables do not obstruct the surgeon or surgical team during the treatment.

The device can include one or more additional magnetic coil tracking devices that may be arranged on a treatment apparatus or treatment-assisting apparatus, in particular on instruments, or on parts of the patient's body, specifically on the pelvis, and which may be situated within the magnetic field or can be introduced into the magnetic field.

A data processing unit can be provided with the device, wherein said data processing unit receives and processes the tracking data. The data processing unit may be part of a computer-assisted medical navigation system. The tracking data can be transmitted to the navigation system either by the aforesaid control device or by wireless data transmission, for example.

In accordance with another aspect of the invention, the device can be provided in combination with an optical tracking system that localizes and tracks optical reference marker arrays. The positional information obtained from magnetic tracking and the positional information obtained from optical tracking can be combined or mutually supplemented in the navigation system. It is then in turn advantageous if at least one of the optical reference marker arrays is arranged in a predetermined and/or known, in particular fixed, positional relationship with respect to the magnetic field generator and/or one of the magnetic coil tracking devices. In the simultaneous tracking which this enables via the electromagnetic and the optical tracking system, the fixed relative positioning of the origins of the two tracking systems allows a transformation onto the other coordinate system, enabling instruments provided with optical and/or electromagnetic tracking means to be simultaneously tracked.

The invention thus relates to a patient positioning device, such as a positioning device for stabilizing the pelvis during a surgical treatment, wherein the patient positioning device is equipped with magnetic tracking units and with a magnetic field generator. The patient positioning device can be an integral part of a surgical navigation system and can communicate its own adjustment and orientation to the navigation computer. The magnetic field generator is part of a tracking device and can detect pelvic movement, surgical instruments and the relative positions of fixing devices that fix the pelvis (e.g., on the frontal plane).

### BRIEF DESCRIPTION OF THE DRAWINGS

The forgoing and other features of the invention are hereinafter discussed with reference to the drawing.

FIG. 1 shows an exemplary medical pelvic positioning and tracking device in accordance with the invention.

### DETAILED DESCRIPTION

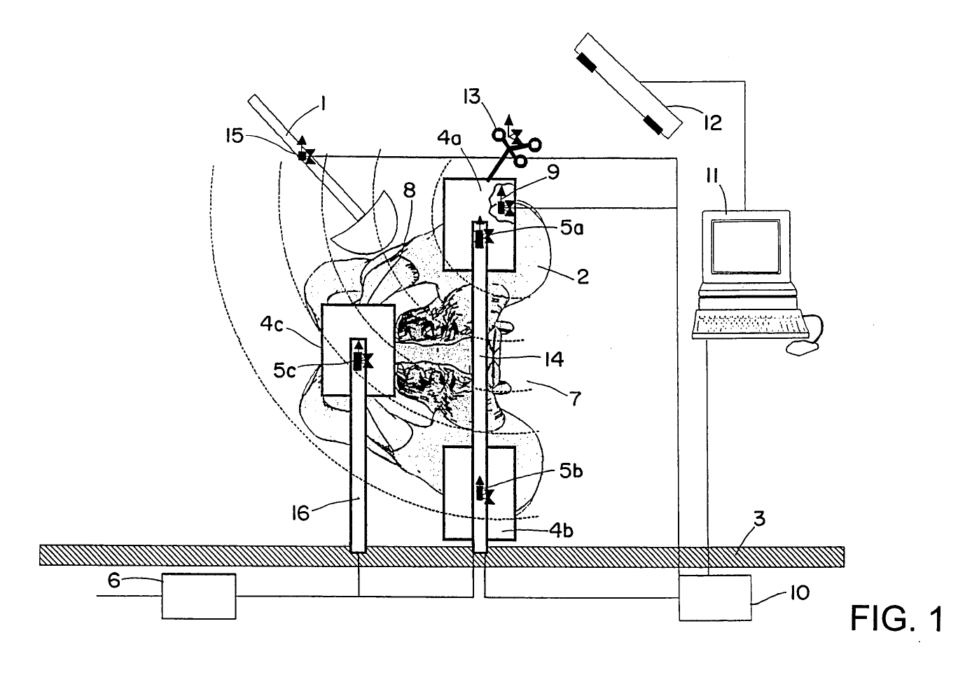

Referring to FIG. 1, a pelvis 2 is shown in a position corresponding to a patient lying on his/her side on a patient table 3. The pelvis 2 is held on its front side by three pelvic support elements 4a, 4b and 4c, two of which (4a and 4b) are movably arranged on a support element holder 14 and another of which (4c) is movably arranged on a support element holder 16, such that they can be adapted to the pelvic anatomy of a patient. The support element holders 14 and 16 are fastened to the patient table 3, wherein it is possible to provide a relative adjustment between the support element holders 14 and 16 in a direction along the length of the table 3.

The pelvic support elements 4a, 4b and 4c can be constituted in the manner of holding cushions that can press onto three points on the pelvis. The three points can be the two pubic tubercles on which the support element 4c is placed, and the left and right front iliac spine on which the two support elements 4b and 4a come to rest. The magnetic field generator 5a (shown only schematically) is accommodated in the pelvic support element 4a and generates a magnetic tracking field, the volume of which is indicated by the reference sign 7. Magnetic coil tracking devices 5b and 5c (also shown only schematically here) are arranged in the pelvic support elements 4b and 4c. Another magnetic coil tracking device 9 is attached to the pelvic bone itself, and the surgical instrument 1 also bears a magnetic coil tracking device 15. The magnetic coil tracking devices 5b, 5c, 9 and 15, together with the magnetic field generator 5a, constitute an exemplary magnetic tracking system.

The magnetic coil tracking devices 5b, 5c, 9 and 15 can in principle be passive resonance coils; active magnetic coil tracking means, however, also can be used. In the present case, the two magnetic coil tracking devices 5b and 5c are supplied with power from the power supply 6, and the corresponding power lines run through the holders 14 and 16. The magnetic field generator 5a also is supplied with power via the power supply 6 using a line through the holder 14. The pelvic tracking device 9 and the instrument tracking device 15 are either resonance coils or are themselves independently supplied with power (for example by small power supply cells).

The instrument 1 can thus be freely moved within the magnetic field 7 and for example guided to the point 8 at which a surgical incision is to be made on the pelvis 2.

A control device 10 can control the magnetic field generator 5a and relay the positional data of the magnetic coil tracking devices 5b, 5c, 9 and 15 to a medical navigation system 11. In this respect, various connecting lines are shown which lead from the magnetic coil tracking devices 5b, 5c, 9 and 15 to the control device 10 and again from the control device 10 to the navigation system 11.

The device as a whole also can comprise an optical tracking system 12 which can localize the position of the pelvic support element 4a via an optical reference marker array 13, said pelvic support element 4a being in a fixed positional relationship with respect to the magnetic field generator 5a.

The device described herein can be used to electromagnetically track the surgical instrument 1 with respect to its position relative to the pelvis 2, in order to achieve a positionally defined treatment preparation (if the instrument 1 is a treatment preparation instrument) or a correct orientation of an implant (if the instrument is an implant holder). The patient lies on the patient table 3 and his/her pelvis 2 is fixed in the device, on the front side shown, via the three pelvic support elements (stabilizers) 4a, 4b and 4c. The three-dimensional position of the magnetic coil tracking devices 5b and 5c relative to the origin at the generator 5a is calculated in accordance with the currently adjusted state of the pelvic positioning device. The field 7 generated by the generator 5a is large and strong enough to track both the two tracking devices 5b and 5c and the tracking devices 1 and 9 on the instrument and pelvis, respectively. The three coordinates defined by the tracking devices 5b and 5c and by the magnetic field generator 5a spatially represent the front pelvic plane as a reference for orientating the instrument 1. Movements by the pelvis 2 relative to the instrument 1 and/or the positioning device can be tracked by the magnetic coil tracking device 9 arranged on the pelvic bone.

The materials for the pelvic support elements and their holdings preferably are selected so as not to interfere with the magnetic field.

With the aid of the positional data from the tracking devices, the navigation system 11 can provide the user with image assistance, wherein the positional data can be transmitted to the navigation system 11 either via the control unit 10 or by wireless transmission.

An additional navigation assistance may be provided by an optical camera tracking system 12, which also may be connected to the navigation system 11. The tracking system 12 comprises a reference array 13 including three reference markers that are arranged either opposite the magnetic field generator 5a and/or opposite one of the tracking devices 5b or 5c, such that the magnetic tracking device as a whole can be simultaneously tracked both electromagnetically and optically. This positioning of the origins of the two tracking systems then allows a transformation into the other coordinate system in each case, in order to additionally allow instruments provided with optical and/or electromagnetic tracking devices to be simultaneously localized and tracked.

The device described herein thus provides optimized electromagnetic tracking in which suitably arranging the field generator ensures stable, secure and precise electromagnetic tracking. This tracking can be supplemented in a simple way by an optical tracking system.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical pelvic positioning and tracking device, comprising:
at least one pelvic support element arranged on a patient support structure; and
a magnetic field generator operative to generate a magnetic field for an electromagnetic tracking system, wherein the magnetic field generator is arranged in or on the at least one pelvic support element.

2. The device according to claim 1, wherein the at least one pelvic support element is a plurality of pelvic support elements, and a first pelvic support element of the plurality of pelvic support elements includes the magnetic field generator, further comprising one or more magnetic coil tracking devices arranged on other pelvic support elements of the plurality of pelvic support elements.

3. The device according to claim 2, wherein the one or more magnetic coil tracking devices are at least one of a passive resonance coil tracking device, or an active magnetic coil tracking device.

4. The device according to claim 3, wherein the active magnetic coil tracking devices comprise an integrated power supply, an external power supply, and/or control unit.

5. The device according to claim 2, further comprising a data processing unit that receives and processes data from the magnetic coil tracking devices.

6. The device according to claim 5, wherein the data processing unit is part of a computer-assisted medical navigation system.

7. The device according to claim 6, further comprising an optical tracking system that is operative to localize and track one or more optical reference marker arrays, wherein positional information obtained from magnetic tracking and positional information obtained from optical tracking are combined or mutually supplemented in the navigation system.

8. The device according to claim 7, wherein at least one of the optical reference marker arrays is arranged in a predetermined and/or known positional relationship with respect to the magnetic field generator and/or one of the magnetic coil tracking devices.

9. The device according to claim 1, wherein the magnetic field generator is arranged, sterilely enclosed, in or on the at least one pelvic support element.

10. The device according to of claim 1, wherein the at least one pelvic support element comprises a support element holder on which the at least one pelvic support element is adjustably arranged.

11. The device according to claim 10, wherein power and/or signal lines for the magnetic field generator and/or active magnetic coil tracking devices are guided on or in the holder.

12. The device according to claim 10, wherein the at least one pelvic support element is a plurality of pelvic support elements, and a position of a first pelvic support element of the plurality of pelvic support elements relative to a second pelvic support element of the plurality of pelvic support elements is adjustable, and a position of the first and second pelvic support elements relative to an origin of the magnetic field generator can be ascertained by tracking a magnetic coil tracking device coupled to the first and/or second pelvic support elements.

13. The device according to claim 1, further comprising a magnetic coil tracking device arranged on a treatment apparatus or a treatment-assisting apparatus, or on parts of a patient's body, said magnetic coil tracking device situated within the magnetic field or introducible into the magnetic field.

14. The device according to claim 13, wherein the treatment apparatus or treatment assisting apparatus is an instrument.
